(19) United States
(12) Patent Application Publication
(10) Pub. No.: US 2026/0272659 A1
(43) Pub. Date: Sep. 17, 2026

(54) SYSTEMS AND METHODS OF RETENTION FOR GLENOSPHERE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Kenneth Konya, Taylor, TX (US); Forrest A. James, Memphis, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/469,538

(22) PCT Filed: Jun. 5, 2024
(86) PCT No.: PCT/US2024/032544

§ 371 Date: Sep. 26, 2025

Related U.S. Application Data
(60) Provisional application No. 63/472,694, filed on Jun. 13, 2023.
(60) Provisional application No. 63/547,435, filed on Nov. 6, 2023.

(51) Int. Cl. A61F 2/30; A61F 2/40; A61F 2/46

(52) U.S. Cl. CPC A61F 2/30749; A61F 2002/30332; A61F 2002/30433; A61F 2002/30495; A61F 2002/30891; A61F 2002/4085; A61F 2002/4629

(57) ABSTRACT

A retention system (140) for coupling a glenosphere (130) to a baseplate (122) in a reverse shoulder arthroplasty including a glenosphere having a retention recess (135) formed in a base of the glenosphere and a retention fastener (141) configured to engage a threaded hole (123) of the baseplate, the retention fastener including a fastener head (142), where the fastener head is configured to be retained within the retention recess. A retention cap (143) can be configured to be coupled within the retention recess of the glenosphere, the retention cap including a body portion (144) having an opening (145) therethrough. The head of the retention fastener can then be configured to be retained within the opening of the body portion to retain the fastener head within the retention recess. Alternatively, the glenosphere can itself include a step (139) that protrudes radially inward from a wall of the retention recess to prevent the retention fastener from passing through the retention recess.

### CROSS-REFERENCE TO RELATED APPLICATIONS

This is a non-provisional of, and claims the benefit of the filing date of, U.S. provisional patent application No. 63/472,694, filed Jun. 13, 2023, entitled “RETENTION FOR GLENOSPHERE,” and U.S. provisional patent application No. 63/547,435, filed Nov. 6, 2023, entitled “RETENTION FOR GLENSOPHERE,” the entirety of which application is incorporated by reference herein.

### FIELD OF THE DISCLOSURE

The present disclosure relates generally to medical implant devices. More particularly, the subject matter disclosed herein relates to configurations of a shoulder arthroplasty implant system.

### BACKGROUND OF THE DISCLOSURE

Shoulder replacement implants are used for restoration of functionalities of shoulders as well as to treat a variety of degenerative conditions and injuries. Shoulder replacement implants typically come in two designs: anatomic and reverse. Anatomic shoulder implants are used for restoration of natural kinematics of a shoulder by replacing its humeral head and glenoid with similarly shaped prosthetic designs that recreate normal anatomy. The anatomic shoulder implant often has a spherical humeral head and a shallow concave glenoid that articulates with the spherical head. As part of an implantation procedure, an anatomic shoulder implant's stem is securely placed down the shaft of the humerus and the spherical head is often fixed to the stem using a mechanical taper press fit. The glenoid prosthetic component (made from a polymer, e.g., ultra-high molecular weight polyethylene (UHMWPE)) is either cemented directly into the remaining intact glenoid or affixed to a metallic tray, which is secured to the native glenoid bone using bone screws, cement, or similar attachment methods.

In the reverse shoulder implant procedure, the implant's spherical surface, sometimes referred to as a glenosphere, is coupled to the remaining intact glenoid, and a concave articular surface is coupled to the humerus. The implant also includes a stem that is securely placed down the shaft of the humerus to support the concave articular surface in position, such as using a mechanical lock to fix the concave articular surface to the stem.

In existing implants having this reverse configuration, it is customary to have a retention screw which maintains the engagement between a tapered base of the glenosphere and a mating taper of the base plate mounted to the glenoid. In many situations, it is further desirable to allow the glenosphere to be repeatably removed for revision purposes. Facilitating the primary alignment of the tapered surfaces and fixation using a secondary fastener both have to be achieved, however, through a single central hole in the glenosphere. As a result, to accommodate these functions, the system often uses a short, retained screw at the most medial side of the glenosphere and open threads laterally on the glenosphere (e.g., at the crest of the sphere). This configuration poses a challenge to have multiple functional areas within one hole/opening.

It is with respect to these and other considerations that the present disclosure may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In accordance with one or more features of the present disclosure, a retention system for coupling a glenosphere to a baseplate in a reverse shoulder arthroplasty is disclosed. The system may include a glenosphere having a retention recess formed in a base of the glenosphere and a retention fastener configured to engage a threaded hole of the baseplate, the retention fastener including a fastener head, where the fastener head is configured to be retained within the retention recess.

In accordance with one or more further features of the present disclosure, a method for coupling a glenosphere to a baseplate in a reverse shoulder arthroplasty is also disclosed. The method may include coupling a head of a retention fastener within a retention recess formed in a base of a glenosphere. The method may further include engaging the retention fastener with threaded hole of a baseplate while the fastener head of the retention fastener is retained within the retention recess.

In any preceding or subsequent example, the glenosphere may include a tapered recess formed in the base of the glenosphere, the tapered recess being configured to be coupled to a tapered protrusion of the baseplate. In some examples, the retention recess is formed at an interior end of the tapered recess.

In any preceding or subsequent example, a retention cap may be configured to be coupled within the retention recess of the glenosphere, the retention cap including a body portion having an opening therethrough. The fastener head of the retention fastener may then be configured to be retained within opening of the body portion to retain the fastener head within the retention recess.

In any preceding or subsequent example, the body portion of the retention cap may include a flange that protrudes radially inward into the opening. In some examples, the fastener head of the retention fastener has a diameter greater than a width of the opening at the ledge such that the head of the retention fastener is prevented from passing through the opening.

In any preceding or subsequent example, the retention cap may include a plurality of prongs extending radially outward from the body portion. In some examples, the plurality of prongs are configured to engage the retention recess of the glenosphere to couple the retention cap to the glenosphere.

In any preceding or subsequent example, the body portion of the retention cap has a height that is greater than a depth of the retention recess. With this configuration, in some examples, the plurality of prongs are configured to flex radially outward upon compression of the body portion within the retention recess.

In any preceding or subsequent examples, the retention recess includes a ledge that protrudes radially inward from a wall of the retention recess. With this configuration, in some examples, the plurality of prongs of the retention cap are configured to engage the ledge to retain the retention cap within the retention recess.

In any preceding or subsequent examples, the glenosphere includes a step that protrudes radially inward from a wall of the retention recess at an interface between the retention recess and the tapered recess. With this configuration, in some examples, the head of the retention fastener may have a diameter greater than a width of the retention recess at the step such that the head of the retention fastener is prevented from passing into the tapered recess.

In any preceding or subsequent examples, the glenosphere includes a threaded opening in communication with the retention recess and configured to be threadedly coupled to an inserter tool. With this configuration, in some examples, the head of the retention fastener may include a threaded outer surface complementary to the threaded opening in the glenosphere such that the retention fastener may be inserted through the threaded opening into the retention recess.

Examples of the present disclosure provide numerous advantages. For example, the present systems and methods provide secure fixation of the glenosphere to the baseplate while also allowing the glenosphere to be repeatably removed for revision purposes.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 illustrates a side view of components of a reverse shoulder replacement implant according to one or more features of the of the presently disclosed subject matter.

FIGS. 2A and 2B illustrate side perspective cutaway views of a retention system for coupling a glenosphere to a baseplate according to one or more features of the of the presently disclosed subject matter.

FIG. 3A is a side perspective view of a retention cap of a retention system according to one or more features of the of the presently disclosed subject matter.

FIG. 3B is a side cutaway view of the retention cap shown in FIG. 3A.

FIGS. 4A and 4B are side cutaway views of a glenosphere of a retention system according to one or more features of the of the presently disclosed subject matter.

FIG. 5 is a side cutaway view of a glenosphere of a retention system according to one or more features of the of the presently disclosed subject matter.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of a glenosphere retention system will now be described more fully herein with reference to the accompanying drawings, in which one or more features of the glenosphere retention system will be shown and described. It should be appreciated that the various features may be used independently of, or in combination, with each other. It will be appreciated that the glenosphere retention system as disclosed herein may be embodied in many different forms and may selectively include one or more concepts, features, or functions described herein. As such, the glenosphere retention system should not be construed as being limited to the specific examples set forth herein. Rather, these examples are provided so that this disclosure will convey certain features of the glenosphere retention system to those skilled in the art.

In accordance with one or more features of the present disclosure, a retention system used to retain the glenoid screw in place is disclosed. The disclosed feature replaces a threaded washer and locking pin configuration that is used in conventional designs. In some examples, the retention system design can help to optimize thread length and head sizing.

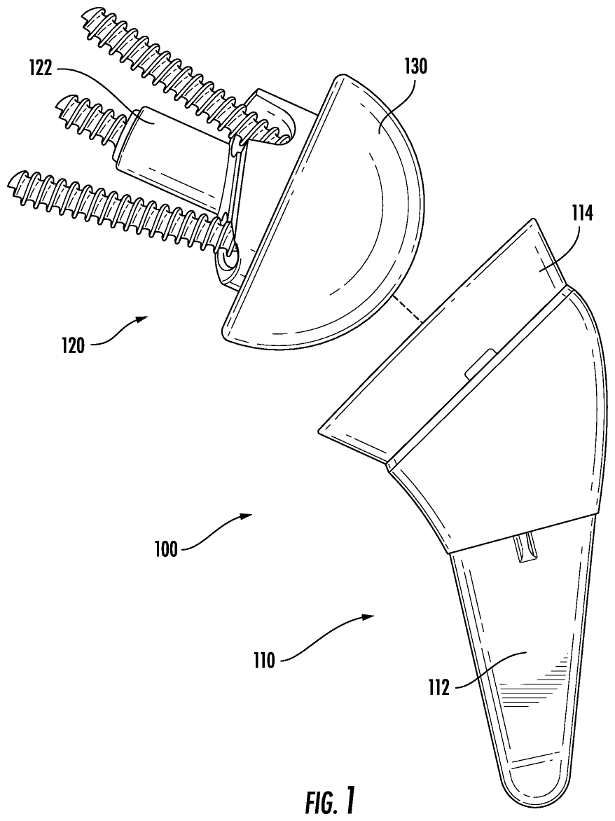

Referring to FIG. 1, the features of the present disclosure provide improvements in the fixation of elements in a reverse shoulder replacement implant, generally designated 100, having a humeral head component 110 including a stem 112 and an articular cup 114 and a glenoid component 120 including a baseplate 122 and a glenosphere 130. In particular, the features of the present disclosure provide improved fixation of the glenosphere 130 to the baseplate 122.

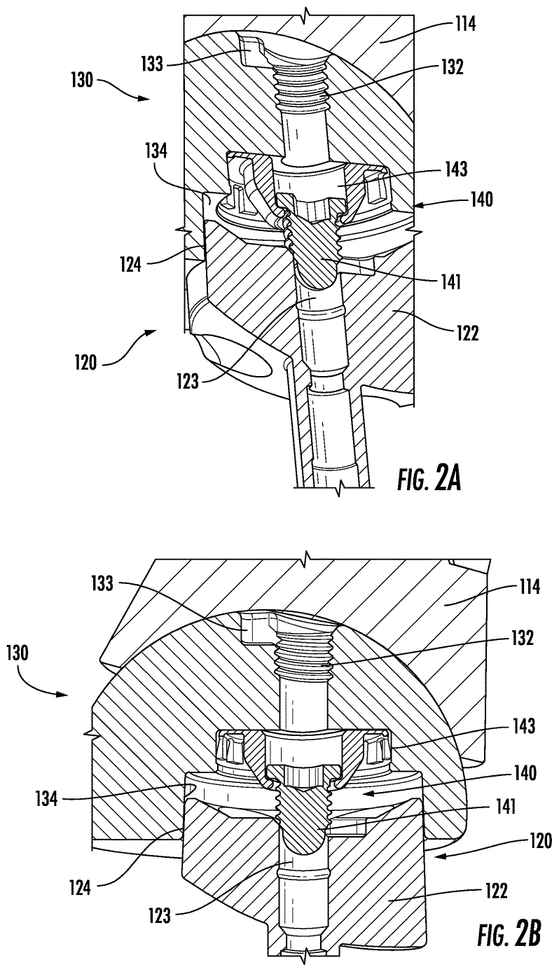

Referring to FIGS. 2A and 2B, in some examples, the glenosphere 130 is attachable to the baseplate 122 using a combination of features. In the illustrated example, initial fixation of the components can be achieved by engaging a tapered protrusion 124 of the baseplate 122 with a tapered recess 134 formed in a base of the glenosphere 130. In some examples, the tapered recess 134 has a shape that substantially corresponds to a shape of the tapered protrusion 124 and that can matingly couple with the tapered protrusion 124 to securely hold the glenosphere 130 in place with respect to the baseplate 122. In some particular examples, the tapered protrusion 124 comprises a Morse taper which can cooperate with the tapered recess 134 to couple the glenosphere 130 to the baseplate 122.

In some examples, assembly of the glenosphere 130 with the baseplate 122 is achieved using an inserter tool. The inserter tool can be attached to the glenosphere by engaging a threaded opening 132 in the glenosphere 130. In addition, in some examples, the inserter tool can include a protrusion that is configured to engage a notch 133 in the glenosphere 130 that is positioned at or near the end of the threaded opening 132, this engagement allowing enhanced rotational control while the components are engaged. Once connected, a user can position the glenosphere 130 such that the tapered recess 134 is aligned with the tapered protrusion 124, and a force can be applied to the inserter tool (e.g., by tapping with a mallet) to secure the glenosphere 130 in place. Further engagement of the elements can be achieved using an impactor according to conventional methods.

In addition, in some examples, fixation of the glenosphere 130 to the baseplate 122 is further achieved using a retention system, generally designated 140, which can include a retention fastener 141 that is configured to be coupled to the glenosphere 130. The retention system 140 can be coupled to the glenosphere 130. The retention fastener 141 can be configured to engage the baseplate 122 of the glenoid component 120 to enhance the coupling of the glenosphere 130 to the baseplate 122. In some examples, the retention fastener 141 has a threaded configuration that corresponds to a threaded hole 123 in the baseplate 122.

In some examples, the retention system 140 further includes a retention cap 143 that is coupled to the retention fastener 141 to secure the retention fastener 141 to the glenosphere 130. In this configuration, the retention cap 143 serves both to couple the retention system 140 to the glenosphere 130 and to act as a washer to distribute the load of the retention fastener 141 when engaged with the baseplate 122. In some examples, the retention cap 143 is composed of a resilient material that is configured to be elastically deformable. In some specific examples, the retention cap 143 is composed of a flexible, implant grade material, including but not limited to 316 stainless steel.

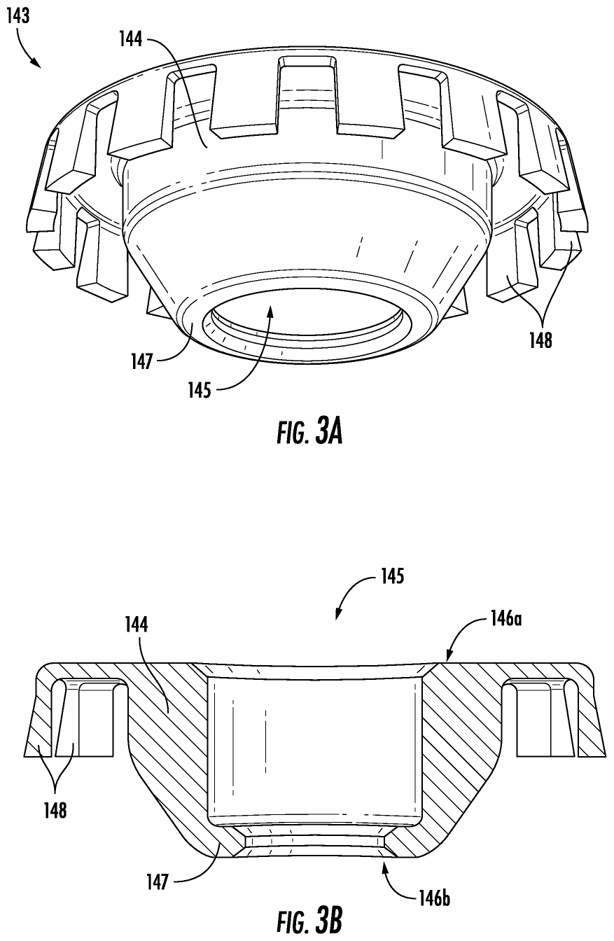

Referring to FIGS. 3A and 3B, in some examples, the retention cap 143 has a body portion 144 having a central opening 145 through which the retention fastener 141 can be received. In some examples, the body portion 144 has a substantially cylindrical shape extending from a first end 146a to a second end 146b substantially opposing the first end 146a. The opening 145 can extend through a center of the substantially cylindrical shape from the first end 146a to the second end 146b. In some examples, the second end 146b of the body portion defines a flange 147 that surrounds the opening 145 and is sized and configured to support a head 142 of the retention fastener 141 thereon.

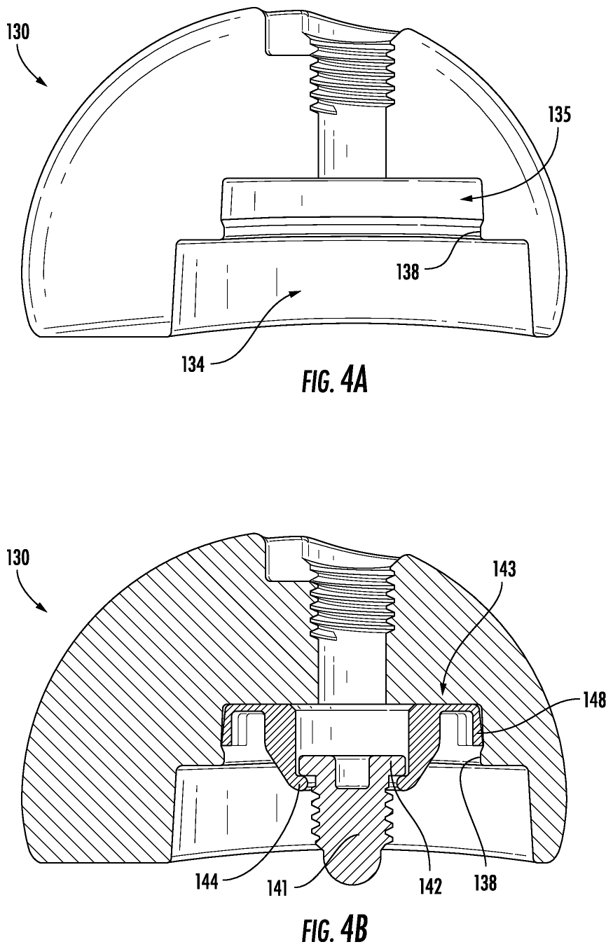

The retention cap 143 can further include a plurality of prongs or fingers 148 that extend from the body portion 144. In some examples, the prongs 148 extend from the first end 146a of the body portion 144. The prongs 148 are configured to engage the glenosphere 130 to retain the retention cap 143 in a substantially fixed position. In some examples, the prongs 148 are configured to be resiliently bendable such that they act as spring elements that resist deflection. Referring to FIGS. 4A and 4B, the glenosphere can include a retention recess 135 within the glenosphere 130 that is sized and configured to receive the retention cap 143 therein. In the illustrated example, the retention recess 135 is formed at an interior end of the tapered recess 134 such that, when the tapered recess 134 is engaged with the tapered protrusion 124 of the glenoid component 120, the retention system 140 is positioned adjacent to the baseplate 122.

In some examples, the retention cap 143 is sized and configured to nest within the retention recess 135. In some specific examples, the retention recess 135 comprises a ledge 138 that protrudes radially inward from a wall of the retention recess 135. In this arrangement, positioning the retention cap 143 within the retention recess 135 can involve positioning the retention fastener 141 within the opening 145 of the retention cap 143 and moving the first end 146a of the retention cap 143 into the retention recess 135 such that the protruding ledge 138 presses the prongs 148 radially inward. As discussed above, the material composition of the retention cap 143 and/or the design and configuration of the prongs 148 can allow the prongs 148 to resiliently flex to accommodate this inward pressure applied by the ledge 138. Once the prongs 148 are inserted past the ledge 138, the prongs 148 can spring back radially outward toward the inner wall of the retention recess 135. In this position, the prongs 148 can be configured to exert a force against the wall of the retention recess 135 to help hold the retention system 140 in place within the retention recess 135. In addition, in some examples, the ledge 138 is configured to engage the prongs 148 of the retention cap 143 when the retention cap 143 is positioned within the retention recess 135. In this way, the ledge 138 can act as a stop to prevent the retention cap 143 from easily backing out of the retention recess 135 (e.g., unless the prongs 148 of the retention cap 143 are again flexed radially inward).

In this configuration, the retention system 140 can be coupled to the glenosphere 130 prior to its initial engagement with the glenoid component 120. As the tapered protrusion 124 of the baseplate 122 is engaged with the tapered recess 134 of the glenosphere 130, the retention fastener 141 can slide within the opening 145 within the retention cap 143 so that it does not impede the alignment and engagement of the tapered protrusion 124 with the tapered recess 134. Upon fixation of the tapered protrusion 124 with the tapered recess 134, a driver can be inserted through the threaded opening 132 of the glenosphere 130, engaged with the retention fastener 141, and operated to couple the retention fastener 141 with the threaded hole 123 of the baseplate 122. In this arrangement, when the retention fastener 141 is coupled to the baseplate 122, such as by engaging with the threaded hole 123 in the baseplate 122, and the retention cap 143 is coupled to the retention fastener, such as via the bearing surface of the flange 147, the engagement of the prongs 148 with the glenosphere 130 completes the chain of connection between the baseplate 122 and the glenosphere 130.

In addition, in some examples, the engagement of the retention fastener 141 with the baseplate 122 can capture the flange 147 therebetween. Further, in some examples, the body portion 144 of the retention cap 143 can have a thickness that is larger than a depth of the retention recess 135. In this configuration, fixation of the retention fastener 141 with the baseplate 122 can compress the body portion 144 within the retention recess 135. Further, in some examples, this compression of the body portion 144 can be configured to cause the prongs 148 to flare radially outwardly and/or axially toward the baseplate 122. This flexion can urge the prongs 148 to engage the wall of the retention recess 135 and/or the ledge 138 to hold the retention cap 143 in position within the retention recess 135. In addition, in some examples, flexion of the retention cap 143 can correspondingly apply tension to the retention fastener 141 to prevent loosening of the retention fastener 141.

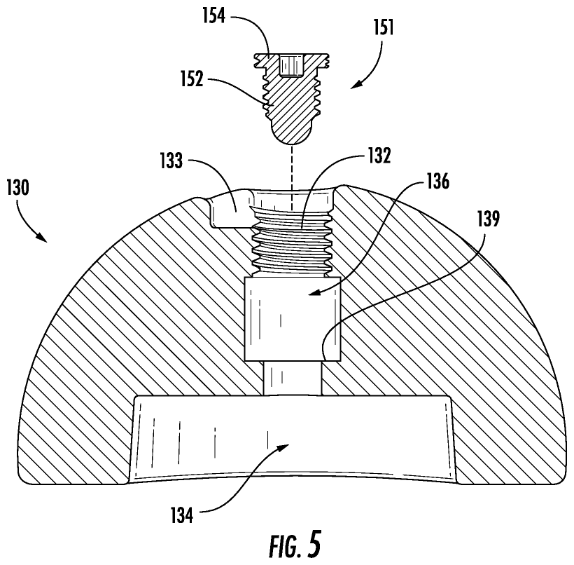

In accordance with one or more further features of the present disclosure, an alternative configuration of a retention mechanism is disclosed. Referring to FIG. 5, in some examples, the retention recess 135 is provided in the form of a stepped opening 136 in communication with the threaded opening 132 in the glenosphere 130. In some examples, the stepped opening 136 narrows at a step 139 that protrudes radially inward from a wall of the stepped opening 136 at an interface between the stepped opening 136 and the tapered recess 134. In this arrangement, the threaded opening 132 can be configured to receive a fastener 151 therethrough. In some examples, the fastener 151 can include a fastener body 152 and a threaded screw head 154 that allows the fastener 151 to pass the threads of the threaded opening 132. The width of the threaded screw head 154 can be wider than the diameter of the stepped opening 136 at the step 139. In this way, the fastener 151 can pass into the stepped opening 136 and extend toward the tapered recess 134, but the threaded screw head 154 of the fastener 151 can become retained on the step 139. In this configuration, the fastener 151 can be positioned to engage the threaded hole 123 of the baseplate 122, and upon fixation of the tapered protrusion 124 with the tapered recess 134, a driver can be inserted through the threaded opening 132 of the glenosphere 130, engaged with the fastener 151, and operated to couple the fastener 151 with the threaded hole 123 of the baseplate 122. In this configuration, the function of the retention cap 143 discussed above is effectively integrated into the internal construction of the glenosphere 130.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one,” “one or more,” and “and/or,” as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

1. A retention system for coupling a glenosphere to a baseplate in a reverse shoulder arthroplasty, the system comprising:
a glenosphere comprising a retention recess formed in a base of the glenosphere; and
a retention fastener configured to engage a threaded hole of the baseplate, the retention fastener comprising a fastener head;
wherein the fastener head is configured to be retained within the retention recess.

2. The retention system of claim 1, wherein the glenosphere comprises a tapered recess formed in the base of the glenosphere, the tapered recess being configured to couple to a tapered protrusion of the baseplate;
wherein the retention recess is formed at an interior end of the tapered recess.

3. The retention system of claim 1, further comprising a retention cap configured to be coupled within the retention recess of the glenosphere, the retention cap comprising a body portion having an opening therethrough;
wherein the fastener head of the retention fastener is configured to be retained within opening of the body portion to retain the fastener head within the retention recess.

4. The retention system of claim 3, wherein the body portion of the retention cap comprises a flange that protrudes radially inward into the opening, wherein the fastener head of the retention fastener has a diameter greater than a width of the opening at the flange such that the fastener head of the retention fastener is prevented from passing through the opening.

5. The retention system of claim 3, wherein the retention cap comprises a plurality of prongs extending radially outward from the body portion, the plurality of prongs being configured to engage the retention recess of the glenosphere.

6. The retention system of claim 5, wherein the body portion of the retention cap has a height that is greater than a depth of the retention recess;
wherein the plurality of prongs are configured to flex radially outward upon compression of the body portion within the retention recess.

7. The retention system of claim 5, wherein the retention recess comprises a ledge that protrudes radially inward from a wall of the retention recess;
wherein the plurality of prongs of the retention cap are configured to engage the ledge to retain the retention cap within the retention recess.

8. The retention system of claim 1, wherein the glenosphere comprises a step that protrudes radially inward from a wall of the retention recess at an interface between the retention recess and the tapered recess;
wherein the fastener head of the retention fastener has a diameter greater than a width of the retention recess at the step such that the fastener head of the retention fastener is prevented from passing into the tapered recess.

9. The retention system of claim 8, wherein the glenosphere comprises a threaded opening in communication with the retention recess and configured to be threadedly coupled to an inserter tool; and
wherein the fastener head of the retention fastener comprises a threaded outer surface complementary to the threaded opening in the glenosphere such that the retention fastener can be inserted through the threaded opening into the retention recess.

10. A method for coupling a glenosphere to a baseplate in a reverse shoulder arthroplasty, the method comprising:
coupling a head of a retention fastener within a retention recess formed in a base of a glenosphere; and
engaging the retention fastener with threaded hole of a baseplate, wherein a fastener head of the retention fastener is retained within the retention recess.

11. The method of claim 10, wherein coupling the fastener head of the retention fastener within the retention recess comprises:
positioning the fastener head of the retention fastener within an opening of a body portion of a retention cap; and
coupling the retention cap within the retention recess of the glenosphere.

12. The method of claim 11, wherein engaging the retention fastener with threaded hole of a baseplate causes flexion of the retention cap, wherein the flexion of the retention cap applies tension to the retention fastener.

13. The method of claim 11, wherein the retention cap comprises a plurality of prongs extending radially outward from the body portion;
wherein coupling the retention cap within the retention recess of the glenosphere comprises engaging the retention recess of the glenosphere with the plurality of prongs.

14. The method of claim 13, wherein the body portion of the retention cap has a height that is greater than a depth of the retention recess;
wherein engaging the retention fastener with threaded hole of the baseplate comprises compressing the body portion of the retention cap within the retention recess; and
wherein compressing the body portion causes the plurality of prongs to flex radially outward against the retention recess.

15. The method of claim 13, wherein the retention recess comprises a ledge that protrudes radially inward from a wall of the retention recess;
wherein coupling the retention cap within the retention recess comprises engaging the plurality of prongs with the ledge to retain the retention cap within the retention recess.

16. The method of claim 10, wherein the glenosphere comprises a step that protrudes radially inward from a wall of the retention recess at an interface between the retention recess and the tapered recess;
wherein coupling the fastener head of the retention fastener within the retention recess comprises positioning the fastener head of the retention fastener against the step.

17. The method of claim 16, wherein the glenosphere comprises a threaded opening in communication with the retention recess and configured to be threadedly coupled to an inserter tool;
wherein the fastener head of the retention fastener comprises a threaded outer surface complementary to the threaded opening in the glenosphere; and
wherein coupling the fastener head of the retention fastener within the retention recess comprises inserting the retention fastener through the threaded opening into the retention recess.
